(19) United States
(12) Patent Application Publication
(10) Pub. No.: US 2016/0361051 A1
(43) Pub. Date: Dec. 15, 2016

(54) SYSTEM FOR THE PARALLEL DELIVERY OF AN ELEMENT INTO THE ESOPHAGEAL MUCOSA

(71) Applicant: Boston Scientific Scimed, Inc., Maple Grove, MN (US)

(72) Inventors: Colby HARRIS, Weston, MA (US); Laura Elizabeth CHRISTAKIS, Worcester, MA (US)

(73) Assignee: Boston Scientific Scimed, Inc., Maple Grove, MN (US)

(21) Appl. No.: 15/175,676

(22) Filed: Jun. 7, 2016

Related U.S. Application Data
(60) Provisional application No. 62/172,979, filed on Jun. 9, 2015.

(51) Int. Cl. A61B 17/00; A61B 17/30; A61B 17/34; A61M 25/06

(52) U.S. Cl. CPC A61B 17/00234; A61M 25/065; A61B 17/30; A61B 17/3478; A61B 17/3468; A61B 2017/00296; A61B 2017/0034; A61B 2017/306; A61M 25/04

(57) ABSTRACT

A system for inserting an element into a target tissue includes an endoscope comprising a working channel extending therethrough and an end cap comprising a cavity extending therein from an open proximal end sized to receive a distal end of the endoscope therein and a closed distal end. The cavity is open to an exterior of the end cap via a window extending laterally through a side wall thereof so that a target tissue may be received within the cavity via the window. The system also comprises a needle sized to be passed through the working channel of the endoscope so that a distal end of the needle is insertable into the cavity thereby, the needle extending longitudinally from a proximal end to a distal end and comprising a lumen for housing an element to be delivered to the target tissue.

### CROSS-REFERENCE TO RELATED APPLICATIONS

This application claims the benefits of priority of U.S. Provisional Application No. 62/172,979, filed on Jun. 9, 2015, the disclosure of which is herein incorporated by reference in its entirety.

### BACKGROUND

For some endoscopic procedures, it may be desired to mark or treat a target area via the insertion of devices such as, for example, markers, seeds and/or anchors. The insertion of these devices in the esophageal mucosa, however, may be difficult due to the thickness of the mucosa.

### SUMMARY

The present disclosure generally relates to a system for inserting an element into a target tissue, comprising an endoscope including a working channel extending therethrough, an end cap including a cavity extending therein from an open proximal end sized and shaped to receive a distal end of the endoscope therein and a closed distal end, the cavity open to an exterior of the end cap via a window extending laterally through a side wall thereof so that a target tissue may be received within the cavity via the window, and a needle sized and shaped to be passed through the working channel of the endoscope so that a distal end of the needle is insertable into the cavity thereby, the needle extending longitudinally from a proximal end to a distal end and including a lumen for housing an element to be delivered to the target tissue.

In an embodiment, the system may further comprise a vacuum source coupled to a proximal end of the endoscope to apply a suction force to the cavity.

In an embodiment, the needle may include a tapered distal tip.

In an embodiment, the needle may include a slit extending along a distal portion thereof, the slit biased in a closed configuration so that a distal end of the lumen is biased closed.

In an embodiment, the needle may be formed of a material that is heat set to be biased closed at a body temperature.

In an embodiment, the system may further comprise a pusher slidably received within the lumen of the needle for pushing the element distally out of the needle and into the target tissue.

The present disclosure also relates to a device for delivering an element to a target tissue. The device comprises an outer catheter extending longitudinally from a proximal end to a distal end and including a channel extending therethrough, a pair of jaws coupled to the distal end of the catheter so that the pair of jaws are movable between an open configuration, in which distal ends thereof are separated from one another to receive a target tissue therebetween and a closed configuration, in which distal ends thereof are moved toward one another to grip the target tissue, and a needle passable through the channel of the outer catheter so that a distal end is extendable into a space between the pair of jaws, the needle extending longitudinally from a proximal end to the distal end and including a first lumen extending therethrough for housing an element to be delivered to the target tissue.

In an embodiment, each of the pair of jaws may include surface features extending along an interior surface thereof for gripping the target tissue between the pair of jaws.

In an embodiment, each of the pair of jaws may be formed of a sheet of metal including edges that are one of bent and curved inward relative to one another.

In an embodiment, proximal ends of the jaws may be pivotally coupled to the distal end of the outer catheter.

In an embodiment, the pair of jaws may be biased in the open configuration, each of the pair of jaws extending along a length that is substantially straight.

In an embodiment, the pair of jaws may be moved between the open and the closed configuration via a control element extending from a proximal end at the proximal end of the outer sheath to a distal end connected to the pair of jaws.

In an embodiment, the needle may include a second lumen extending therethrough for injecting a fluid into the target tissue.

In an embodiment, the needle may include a slit extending along a distal portion thereof, the slit biased in a closed configuration so that a distal opening of the first lumen is biased closed.

In an embodiment, the device may further comprise a pusher element slidably received within the first lumen for pushing the element distally out of the lumen into the target tissue.

The present disclosure is also directed to a method for delivering an element to a target portion of an esophageal mucosa, comprising inserting a device through the working channel of an endoscope such that a pair of jaws coupled to a distal end of an outer catheter is adjacent the target portion of the esophageal mucosa, moving the pair of jaws from a closed configuration to an open configuration in which distal ends thereof are separated to receive the target portion, moving the pair of jaws to the closed configuration by drawing distal ends thereof toward one another to grip the target portion therebetween, sliding a needle distally through a channel of the outer catheter so that a distal end of the needle extends into a space between the pair of jaws and into the target portion, and pushing an element housed within a first lumen of the needle distally out of the needle into the target portion of the esophageal mucosa such that the element extends substantially parallel to a wall of the esophageal mucosa.

### BRIEF DESCRIPTION

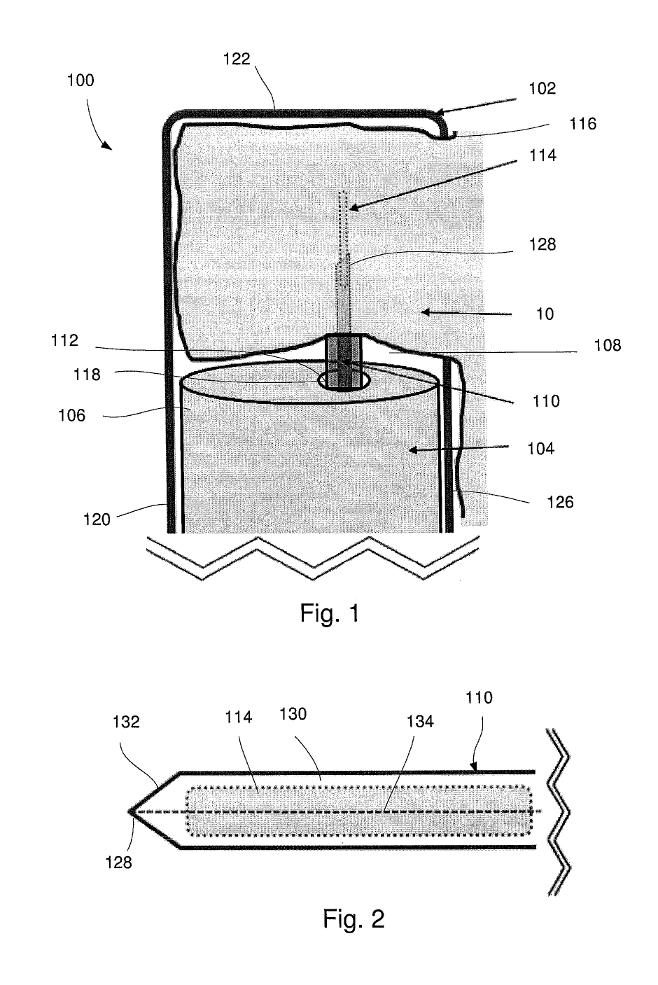

FIG. 1 shows a partial longitudinal cross-sectional view of a distal portion of a system according to an exemplary embodiment of the present disclosure;

FIG. 2 shows a longitudinal side view of a needle according to the system of FIG. 1;

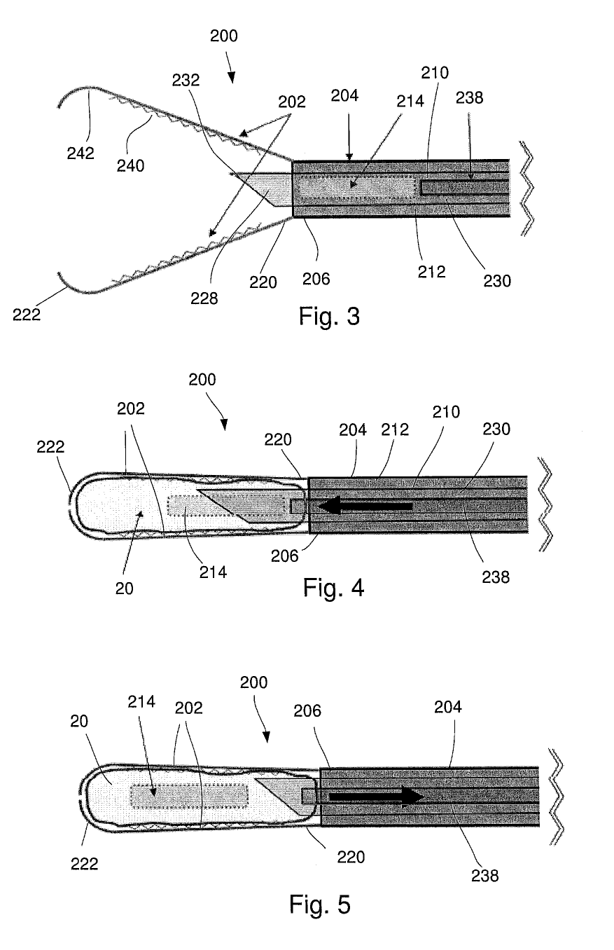

FIG. 3 shows longitudinal side view of a device according to another exemplary embodiment of the present disclosure, in a first position;

FIG. 4 shows a longitudinal side view of the device of FIG. 3, in a second position;

FIG. 5 shows a longitudinal side view of the device of FIG. 3, in a third position;

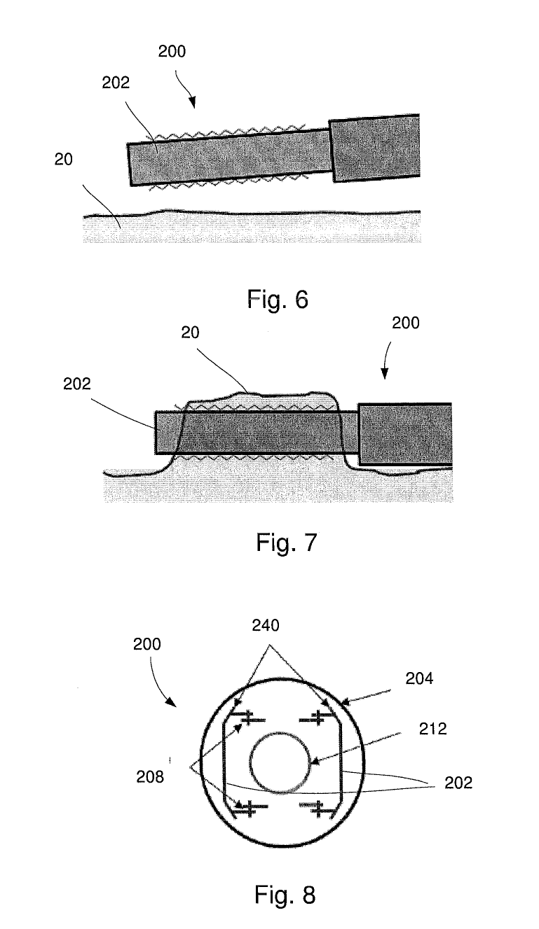

FIG. 6 shows another longitudinal side view of the device of FIG. 3, in which jaws thereof extend substantially parallel to a target tissue;

FIG. 7 shows the longitudinal side view of the device of FIG. 6, in which the jaws are gripping the target tissue;

FIG. 8 shows a lateral cross sectional view of the device of FIG. 3;

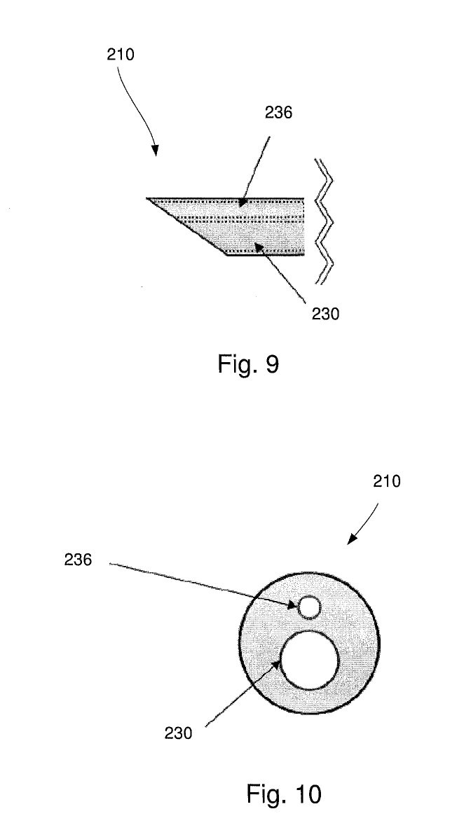

FIG. 9 shows a longitudinal side view of a distal portion of a needle according to the system of FIG. 3; and

FIG. 10 shows a lateral cross-sectional view of the needle of FIG. 9.

### DETAILED DESCRIPTION

The present disclosure may be further understood with reference to the following description and the appended drawings, wherein like elements are referred to with the same reference numerals. The present disclosure relates to systems for delivering an element into a target tissue and, in particular, relates to systems for inserting elements such as markers, radioactive seeds and anchors, to an esophageal mucosa via a parallel approach. Exemplary embodiments of the present disclosure describe a device for gathering an esophageal mucosa so that an element may be inserted into the esophageal mucosa via a parallel approach. It should be noted that the terms “proximal” and “distal,” as used herein, are intended to refer to a direction toward (proximal) and away from (distal) a user of the system.

As shown in FIGS. 1 and 2, a system 100 according to a first exemplary embodiment of the present disclosure comprises an end cap 102 mountable over a distal end 106 of an endoscope 104 to gather target tissue 10 (e.g., esophageal mucosa) within a cavity 108 thereof. Upon gathering the target tissue 10 in the cavity 108, a delivery needle 110 housing an element 114 such as, for example, a marker, radioactive seed or anchor, may be passed through a working channel 112 of the endoscope 104 and into the cavity 108 to insert the element 114 into the captured target tissue 10. The end cap 102 includes a lateral window 116 which extends through a side wall of the end cap 102 to face radially outward from the end cap 102. A portion of target tissue 10 (e.g., esophageal mucosa) may be suctioned into the cavity 108 via the lateral window 116 as will be described in more detail below. Thus, as the needle 110 is moved distally through working channel 112 into the cavity 108, the needle 110 penetrates the target tissue 10 substantially parallel to a wall thereof—i.e., the needle 110 is moving substantially parallel to a wall of the portion of the tissue not drawn into the cavity 108. This parallel approach permits a user, such as a surgeon or other physician, to control the depth of insertion of the element 114 into the target tissue 10. Dimensions of the cavity 108 and/or a level of suction applied thereto may be selected to achieve the desired depth of injection of the element 114. That is, a distance between a portion of the wall of the end cap 102 opposite the window and the lateral window 116 defines a maximum extent to which the tissue may be drawn into the cavity 108. As would be understood by those skilled in the art, the position of the needle 110 along an axis between the lateral window 116 and the portion of the wall of the end cap 102 opposite the window 116 defines a maximum depth to which the element 114 may be inserted (i.e., when the needle 110 is penetrated halfway through the thickness of the tissue drawn into the cavity 108). Thus, the depth of insertion of the element 114 may be controlled by selecting an end cap 102 having a cavity 108 with the desired dimensions. A user, however, may also control a depth of insertion of the element 114 by controlling the suction to adjust a depth to which the tissue is drawn into the cavity 108.

As would be understood by those skilled in the art, the endoscope 104 may be any sufficiently flexible endoscope sized and shaped for insertion through a patient's mouth into the esophagus or via another naturally occurring bodily orifice into its corresponding body lumen. The endoscope 104 includes the working channel 112 extending longitudinally therethrough from a proximal opening to a distal opening 118. The endoscope 104 may also include a second channel through which a suctioning force may be applied. Alternatively, however, the suction force may also be applied through the working channel 112 via a vacuum source coupled to a proximal end of the endoscope 104.

The end cap 102 extends longitudinally between its proximal end 120 which is configured to engage the distal end 106 of the endoscope 104 and its distal end 122. An opening 124 at the proximal end 120 of the end cap 102 is sized and shaped to receive the distal end 106 of the endoscope 104 with the end cap 102 engaging the endoscope 104 via a friction fit or any other suitable mechanical engagement. The end cap 102 defines a cavity 108 extending therein from the opening 124 at the proximal end 120 to a closed distal end 122. The window 116 extends laterally through a side wall 126 of the end cap 102 so that, when the end cap 102 is coupled to the endoscope 104, the cavity 108 is open to an exterior of the end cap 102 via the window 116. Thus, when the end cap 102 is mounted over the distal end 106 of the endoscope 104, negative fluid pressure applied through the endoscope 104 applies a suction force through the cavity 108, drawing target tissue 10 through the window 116 into the cavity 108.

The delivery needle 110 is preferably sized and shaped for insertion through the working channel 112 of the endoscope 104. The needle 110 extends longitudinally from a proximal end (not shown) to a distal end 128. The delivery needle 110 also includes a lumen 130 extending therethrough for housing the element 114. A control member such as, for example, a piston or other pushing element is passed through the lumen 130 to push the element 114 beyond the distal end 128 out of the lumen 130 into the target tissue 10. The distal end 128 of the needle 110 includes a tissue piercing tip such as the exemplary tapered distal tip 132 shown in this embodiment.

In one exemplary embodiment, as shown in FIG. 2, the needle 110 includes a slit 134 extending along distal portion thereof to the distal end 128. The slit 134 according to this embodiment is biased toward a closed configuration, which holds the distal end 128 closed to prevent the element 114 housed therein from the passing distally out of the lumen 130 until it is desired to deploy the element 114. That is, the bias of the slit 134 holds the tip 132 closed to seal the lumen 130 until the user forces the tip 132 open by pushing the control member distally to force the element 114 out of the needle 110. The needle 110 may be formed of material such as, for example, nitinol, which may be heat set to be biased toward the closed position at body temperature. When it is desired to deploy the element 114, the control element may be moved distally relative to the needle 110 to push the element 114 against the distal end 128, forcing the slit 134 to open. Once the slit 134 has moved to the open configuration, the element 134 is pushed distally therepast to be inserted into the target tissue 10.

According to an exemplary method using the system 100, the end cap 102 is mounted on the distal end 106 of the endoscope 104 and the distal end 106 of the endoscope 104 is then inserted through the patient's mouth into the esophagus until the end cap 102 is adjacent a target portion of tissue 10. Once the endoscope 104 has been positioned as desired with the lateral window 116 facing the target tissue 10, negative pressure is applied through the endoscope 104 and thereby the end cap 102, to draw the target tissue 10 into the cavity 108 via the window 116. In one embodiment, the suction force may be applied through the working channel 112, through a space, for example, annularly surrounding the needle 110 received therein. In another embodiment, suction force may be applied through the endoscope 104 via a second channel (not shown).

Once the target tissue 10 (e.g., a target portion of the esophageal mucosa) has been suctioned into the cavity 108 of the end cap 102 to the desired extent, the needle 110 is moved distally through the working channel 112 until the distal end 128 of the needle 110 extends distally past the distal end 106 of the endoscope into the cavity 108. The distal tip 132 of the needle 110 pierces the target tissue 10 received within the cavity and the needle 110 is moved distally until the tip 132 penetrates to the desired depth in the tissue 10. The element 114 is then pushed distally past the distal end 128 of the needle 110 until the element 114 is inserted in the target tissue 10. After the element 114 has been inserted in the target tissue 10, the needle 110 is retracted and the suction force is deactivated so that the target tissue 10 reverts to its original position. As described above, the parallel approach—insertion of the element 114 substantially parallel to a wall of the esophageal mucosa—permits the user to control a depth of insertion of the element 114. The user may also select a desired level of suction force and/or end cap 102 having a desired cavity 108 size to further control a depth of insertion. Additional elements 114 may be inserted, as desired, in the same manner as described above. Upon insertion of the element(s) 114, as desired, the endoscope 104 may be removed from the body.

As shown in FIGS. 3-8, a device 200 for inserting an element 214 into a target tissue 20 (e.g., esophageal mucosa) via a parallel approach comprises a pair of jaws 202 connected to a distal end 206 of an outer catheter 204 and a needle 210 passable through the outer catheter 204 to deliver the element 214. The device 200 may be inserted through, for example, a working channel of an endoscope, to the target tissue 20, so that the jaws 202 extend substantially parallel to the target tissue 20, as shown in FIGS. 6 and 7. The jaws 202 are movable between an open tissue receiving configuration in which tissue may be received therebetween and a closed tissue gripping configuration in which tissue is gripped therebetween. Once the jaws 202 have gripped the target tissue 20, the needle 210 is moved distally through the outer catheter 204, between the jaws 202 and into the target tissue 20 gripped thereby. Teeth 240 along jaws 202 may be configured such that, when the jaws 202 are rested on a surface of the target tissue 20 in a parallel configuration, the teeth 240 grab the target tissue 20 together as the jaws 202 are moved toward the closed configuration. Thus, the target tissue 20 is gripped between the jaws 202. The needle 210 may then be extended distally out of the catheter 204 into the gripped tissue 20 and the element 214 may be pushed distally through the needle 210 until the element 214 is inserted into the target tissue 20.

As shown in FIGS. 3-5 and 8, the catheter 204 extends from a proximal end (not shown) to a distal end 206 and includes a channel 212 extending longitudinally therethrough. Each of the jaws 202 extends from a proximal end 220 coupled to the distal end 206 of the catheter 204 to a distal end 222. The jaws 202 are coupled to opposing sides of the distal end 206 of the catheter 204 such that the jaws 202 are movable between the open configuration, in which the distal ends 222 are separated from one another, and the closed configuration, in which the distal ends 222 are moved toward one another to grip the tissue therebetween. In one embodiment, the jaws 202 are coupled to the catheter 204 and are biased toward the open configuration. Thus, when the jaws 202 are within the working channel of the endoscope through which the device 200 is passed to the target tissue 20, a surface of the working channel holds the jaws 202 in the closed configuration. When the jaws 202 are moved distally past the distal end of the endoscope (or other delivery instrument), the jaws 202 are permitted to revert to the biased open configuration. When the jaws 202 are positioned about the target portion of tissue 20, the endoscope may be advanced over the jaws 202 to draw the jaws 202 together into the closed tissue gripping configuration. In another embodiment, the jaws 202 are pivotally coupled to the distal end 206 of the outer catheter 204 and are movable between the open and closed configurations via a control element extending from a proximal end connected to a proximal portion of the catheter 204 such that it is accessible to a user and a distal end connected to the jaws 202. Thus, the user may actuate the control element to move the jaws 202 between the open and closed configurations.

In one example, the jaws 202 are coupled to the distal end 206 of the outer catheter 204 via pinned hinges 208, as shown in FIG. 8. The pinned hinges 208 permit the jaws 202 to pivot between the open and closed configurations. The pinned hinges 208 may also include a spring mechanism biasing the jaws 202 toward the open configuration. As would be understood by those skilled in the art, the jaws 202 may be coupled to the distal end of the outer catheter 204 in any of a variety of ways, so long as the jaws 202 may be moved between the open tissue receiving configuration and the closed tissue gripping configuration.

Each of the jaws 202 may include teeth 240, or other surface features such as projections or protrusion, along an interior surface 242 thereof—i.e., a surface of the jaw 202 which faces the opposing jaw 202—for aiding in the gripping of tissue therebetween. In one embodiment, each of the jaws 202 may be formed of a sheet of metal stamped with teeth 240, so that the jaws 202 extend substantially straight with longitudinal edges 244 of the jaws 202 being bent inwards—toward the opposing jaw 202—to aid in tissue gathering. The distal ends 222 may also be curved inward.

The needle 210 is sized and shaped to be inserted through the channel 212 of the catheter 204 and extends from a proximal end to a distal end 228 and includes a first lumen 230 extending therethrough for housing the element 214 to be inserted into the target tissue 20. The distal end 228 includes a tissue piercing tip such as the tapered tip 232 shown. In one exemplary embodiment, the needle 210 is substantially similar to the needle 110 described above in regard to the system 100. In another exemplary embodiment, as shown in FIGS. 9-19, the needle 210 includes a second lumen 236 extending longitudinally therethrough for delivering fluid (e.g., saline) to the target tissue 20. The fluid may be injected into the target tissue 20, between the mucosa and the submucosa, to raise a target section of the mucosa so that it may be gripped by the jaws 202 parallel to the wall of the tissue 20. Once the target tissue 20 is raised, the target tissue 20 may be better gripped between the jaws 202. This embodiment of the needle 210 may also include a slit extending along a distal portion thereof as described in regard to the slit 134 of the needle 110. In particular, the slit may extend along a wall of the first lumen 230 so that the first lumen 230 is spreadable between a biased closed configuration in which edges of the slit are in contact with one another to prevent passage of the element 214 out of the first lumen 230 and an open configuration, in which, as the element 214 is forced distally against the tip 232 (via, e.g., a pusher element 238), the slit opens to permit the element 214 to be pushed distally out of the needle 210 into the target tissue 20.

According to an exemplary method using the system 200, the device 200 is inserted through a body lumen to location adjacent to the target tissue 20—e.g., a portion of the esophageal mucosa—via a working channel of an endoscope, a distal end of which is positioned adjacent the target tissue 20. The device 200 is moved distally through the working channel 200 until the jaws 202 are moved distally past the distal end of the endoscope and revert to the open configuration, as shown in FIGS. 3 and 6. In one embodiment, movement of the jaws 202 distally past the distal end of the endoscope permits the jaws to revert to a biased open configuration to receive target tissue 20 therebetween. In another embodiment, the jaws 202 may be moved to the open configuration via a proximal end of a control element connected to the jaws 202.

In one exemplary embodiment, the jaws 202 may be rested against a surface of the target tissue 20 so that, as the jaws 202 are moved toward the closed configuration, teeth 240 along the interior of the jaws 202 grip the target tissue 20 therebetween. As described above, a longitudinal edge of each of the jaws 202 may be bent inward—toward the opposing jaw—such that the portion of tissue resting thereagainst is gripped thereby as the jaws 202 are moved toward the closed configuration, aiding in the gripping of tissue between the jaws 202.

In another exemplary embodiment, the needle 210 may have two lumens 230, 236 such that the a fluid may be delivered to the target tissue 20 via one of the lumens 230, 236 prior to gripping the target tissue 20. The needle 210 is passed through the channel 212 of the catheter 204 so that the tapered tip 232 pierces the target tissue 20. A portion of the needle 210 may be moved against the interior surface of the channel 212 to direct the needle 210 into the target tissue 20. A fluid for raising the target tissue 20 is then injected into the target tissue 20 via the second lumen 236. The needle 210 may then be removed from the tissue and drawn into the channel 212 of the outer catheter 204. Once the target tissue 20 has been raised, the jaws 202 are positioned on opposite sides of the raised tissue and are then moved to the closed configuration to grip the target tissue 20, as shown in FIGS. 7.

Where the jaws 202 are biased in the open configuration, the jaws 202 may be closed by drawing the jaws 202 slightly proximally into the working channel of the endoscope or by advancing the endoscope distally over the jaws 202. The jaws 202 are configured—e.g., substantially straight—so that drawing even just a small portion of the proximal ends 220 of the jaws 202 into the working channel moves the jaws 202 toward the closed configuration to grip the target tissue 20 therebetween. Alternatively, the jaws 202 may be moved toward the closed configuration via a control element connected to the jaws 202 by any known mechanism as described above.

When the target tissue 20 has been gripped by the jaws 202, the needle 210 is moved distally out of the catheter 204 between the jaws 202 to penetrate the target tissue 20 gripped by the jaws 202. The pusher element 238 is then moved distally relative to the needle 210, as shown in FIG. 4, to push the element 214 distally out of the needle 210 (e.g., out of the first lumen 230) and into the target tissue 20. Similarly to the system 100, the element 214 is inserted into the target tissue 20, substantially parallel to a wall thereof. Upon insertion of the element 214 in the target tissue 20, the needle 210 may be retracted, as shown in FIG. 5 and the jaws 202 moved to the open configuration to release the target tissue 20. Once the target tissue 20 has been released the device 200 may be removed from the body.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

What is claimed is:

1. A system for inserting an element into a target tissue, comprising:
an endoscope comprising a working channel extending therethrough;
an end cap comprising a cavity extending in the end cap from an open proximal end of the end cap, sized to receive a distal end of the endoscope, to a closed distal end of the end cap, the cavity open to an exterior of the end cap via a window extending laterally through a side wall of the end cap so that a target tissue may be received within the cavity via the window; and
a needle sized to be passed through the working channel of the endoscope so that a distal end of the needle is insertable into the cavity, the needle extending longitudinally from a proximal end of the needle to the distal end of the needle, and the needle comprising a lumen for housing an element to be delivered to the target tissue.

2. The system of claim 1, further comprising a vacuum source coupled to a proximal end of the endoscope to apply a suction force to the cavity.

3. The system of claim 1, wherein the needle comprises a tapered distal tip.

4. The system of claim 1, wherein the needle comprises a slit extending along a distal portion of the needle, the slit biased to a closed configuration so that a distal end of the lumen of the needle is closed.

5. The system of claim 4, wherein the needle is formed of a material that is heat set to be biased closed at a body temperature.

6. The system of claim 1, further comprising a pusher slidably received within the lumen of the needle for pushing the element distally out of the needle and into the target tissue.

7. A device for delivering an element to a target tissue, comprises:
an outer catheter extending longitudinally from a proximal end to a distal end and comprising a channel extending through the outer catheter;
a pair of jaws coupled to the distal end of the catheter so that the pair of jaws are movable between an open configuration, in which distal ends of the pair of jaws are separated from one another to receive a target tissue between the pair of jaws, and a closed configuration, in which the distal ends of the pair of jaws are proximate one another to grip the target tissue; and
a needle passable through the channel of the outer catheter so that a distal end of the needle is extendable into a space between the pair of jaws, the needle extending longitudinally from a proximal end of the needle to the distal end of the needle and comprising a first lumen extending through the needle for housing an element to be delivered to the target tissue.

8. The device of claim 7, wherein each of the pair of jaws comprises surface features extending along an interior surface thereof for gripping the target tissue between the pair of jaws.

9. The device of claim 7, wherein each of the pair of jaws is formed of a sheet of metal including edges that are one of bent and curved inward relative to one another.

10. The device of claim 7, wherein proximal ends of the jaws are pivotally coupled to the distal end of the outer catheter.

11. The device of claim 7, wherein the pair of jaws are biased to the open configuration, and each of the pair of jaws has a substantially straight portion.

12. The device of claim 7, wherein the pair of jaws are moved between the open configuration and the closed configuration via a control element extending from a proximal end of the control element at the proximal end of the outer sheath, to a distal end of the control element connected to the pair of jaws.

13. The device of claim 7, wherein the needle comprises a second lumen extending through the needle for injecting a fluid into the target tissue.

14. The device of claim 7, wherein the needle comprises a slit extending along a distal portion of the needle, the slit biased in a closed configuration so that a distal opening of the first lumen is closed.

15. The device of claim 7, further comprising a pusher element slidably received within the first lumen for pushing the element distally out of the first lumen into the target tissue.

16. A method for delivering an element to a target portion of an esophageal mucosa, comprising:
inserting a device through a working channel of an endoscope such that a pair of jaws coupled to a distal end of an outer catheter is adjacent the target portion of the esophageal mucosa;
moving the pair of jaws from a closed configuration to an open configuration in which distal ends of the pair of jaws are separated to receive the target portion;
moving the pair of jaws to the closed configuration by drawing the distal ends of the pair of jaws toward one another to grip the target portion between the pair of jaws;
sliding a needle distally through a channel of the outer catheter so that a distal end of the needle extends into a space between the pair of jaws and into the target portion; and
pushing an element housed within a first lumen of the needle distally out of the needle into the target portion such that the element extends substantially parallel to a wall of the esophageal mucosa.

17. The method of claim 16, further comprising:
piercing the target portion of the esophageal mucosa via the distal end of the needle prior to moving the pair of jaws to the closed configuration and injecting a fluid through a second lumen of the needle to raise the mucosa from submucosa so that the target portion may be received between the pair of jaws.

18. The method of claim 16, wherein pushing the element distally out of the first lumen of the needle comprises pushing open a slit in the needle.

19. The method of claim 16, wherein the element is one of an endoscopic marker, a radioactive seed and an anchor.

20. The method of claim 16, wherein proximal ends of each of the pair of jaws are pivotally coupled to the distal end of the outer catheter.
